(19) United States
(12) Patent Application Publication
(10) Pub. No.: US 2025/0009648 A1
(43) Pub. Date: Jan. 9, 2025

(54) COMPOSITIONS AND METHODS FOR TENDON REPAIR

(71) Applicant: MAYO FOUNDATION FOR MEDICAL EDUCATION AND RESEARCH, Rochester, MN (US)

(72) Inventors: Atta Behfar, Rochester, MN (US); Matthew T. Houdek, Rochester, MN (US)

(21) Appl. No.: 18/709,915

(22) PCT Filed: Nov. 16, 2022
(86) PCT No.: PCT/US2022/050121

§ 371 Date: May 14, 2024

Related U.S. Application Data
(60) Provisional application No. 63/279,839, filed on Nov. 16, 2021.

(51) Int. Cl. A61K 9/00; A61K 9/19

(52) U.S. Cl. CPC A61K 9/0024; A61K 9/19

(57) ABSTRACT

A composition includes a purified exosome product (PEP) and a pharmaceutically acceptable carrier that includes a supportive matrix. The supportive matrix can include a collagen scaffold, a tissue sealant, or a fibrin sealant. A method of repairing damaged tendon tissue generally includes applying a composition that includes PEP and a pharmaceutically acceptable carrier to damaged tendon tissue. In one or more embodiments, the composition applied to the damaged tendon tissue includes a supportive matrix.

### CROSS-REFERENCE TO RELATED APPLICATIONS

This application claims the benefit of U.S. Provisional Application Ser. No. 63/279,839, filed on Nov. 16, 2021, which is incorporated by reference herein in its entirety.

### SUMMARY

This disclosure describes, in one aspect, a composition that generally includes a purified exosome product (PEP) and a pharmaceutically acceptable carrier that includes a supportive matrix.

In one or more embodiments, the PEP includes spherical or spheroid exosomes having a diameter no greater than 300 nm.

In one or more embodiments, the PEP includes spherical or spheroid exosomes having a mean diameter of 110 nm±90 nm. In one or more of these embodiments, wherein the PEP includes spherical or spheroid exosomes having a mean diameter of 110 nm±50 nm. In one or more of these embodiments, the PEP includes spherical or spheroid exosomes having a mean diameter of 110 nm±30 nm.

In one or more embodiments, the PEP includes from 1% to 20% CD63⁻ exosomes and from 80% to 99% CD63⁺ exosomes. In one or more of these embodiments, the PEP includes at least 50% CD63⁻ exosomes.

In one or more embodiments, the PEP includes from 1×10¹¹ PEP exosomes to 1×10¹³ PEP exosomes. In one or more of these embodiments, the PEP includes from 1×10¹² PEP exosomes to 1×10¹³ PEP exosomes.

In one or more embodiments, the supportive matrix includes a collagen scaffold. In one or more of these embodiments, the collagen scaffold includes type I fibrillar collagen.

In another aspect, this disclosure describes a method of treating injured tendon tissue, the method including applying a composition described herein to injured tendon tissue.

In one or more embodiments, the composition is applied in an amount effective to decrease adhesion production compared to injured tendon tissue treated without the composition. In one or more embodiments, the composition is applied in an amount effective to increase the ratio of type I to type III collagen compared to injured tendon tissue treated without the composition.

In one or more embodiments, the composition is applied in an amount effective to produce more organized collagen architecture compared to injured tendon tissue treated without the composition.

In one or more embodiments, the injured tendon tissue includes disruption of a tendon. In one or more of these embodiments, the disruption of the tendon includes rupture of the tendon. In one or more of these embodiments, the rupture of the tendon includes an Achilles tendon rupture.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Surgical technique demonstrating the rabbit positioned prone with hindlimb prepped and draped. (A) A 2-centimeter (cm) incision centered 1.5 cm proximal to calcaneal tubercle was performed. (B) The paratenon was incised and the flexor digitorum superficialis (FDS) was identified and isolated. (C) The Achilles tendon was identified and isolated. (D) Tenotomy was made through the Achilles tendon. (E) A modified Kessler core suture was performed in all groups. (F) The scaffold was placed at the tenotomy site for Group 2 and Group 3 prior to final suture tightening. (G) The incision was closed with absorbable suture. (H) and (I) the hindlimb was placed in a hip-spica like cast at a 150° angle for 3-6 weeks.

FIG. 2. Evaluation of tendon repair. (A) Load to failure at three weeks and six weeks for each group. (B) Ultimate tensile strength at three weeks and six weeks for each group. (C) Cross-sectional area decreased in the PEP-treated groups by six weeks (p=0.04). (D) Young's modulus was greater with PEP-treated groups (p=0.01) and increased over time (p≤0.03).

FIG. 3. Evaluation of tendon repair. (Left) Young's modulus in relation to cross-sectional area (labeled “CSA”) for each group. (Right) Ultimate tensile strength in relation to cross-sectional area (labeled “CSA”) for each group. There was a significant interaction between groups in relation to Young's modulus and cross-sectional area (p=0.03) in favor of greater stiffness per cross-sectional area for PEP-treated groups versus control groups. There was no significant interaction between groups with regard to ultimate tensile strength and cross-sectional area (p-0.84).

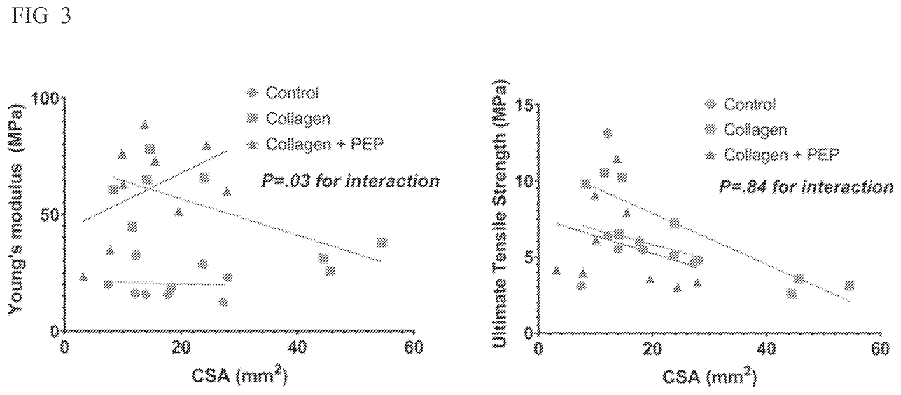

FIG. 4A. Trichrome staining of specimens from each group at each endpoint as well as normal contralateral, untreated tendon.

FIG. 4B. Hematoxylin and eosin (H&E) staining of specimens from each group at each endpoint as well as normal contralateral, untreated tendon. Images show more organized, denser collagen with less peripheral adhesions in the PEP-treated group closer resembling normal tendon.

FIG. 5. Evaluation of tendon repair (A) Macroscopic adhesion grading at three weeks and six weeks for each group. (B) Microscopic adhesion grading at three weeks and six weeks for each group. Group 3 demonstrated significantly less adhesions both macroscopically (p=0.0006) and microscopically (p=0.0062).

FIG. 6. Images depicting tendon adhesions following dissection at the six-week time point. Adhesions were macroscopically greater in the control group (left) and collagen-only group (center) compared to the PEP+collagen group (right) (p=0.0006).

FIG. 7. Immunohistochemical evaluation of tendon repair. (A) Immunohistochemical staining against Type I collagen for specimens from each group as well as normal contralateral, untreated tendon. (B) Immunohistochemical staining against Type III collagen for specimens from each group as well as normal contralateral, untreated tendon. Images show an increase in stain intensity for Type I collagen and decreased stain intensity for Type III collagen with PEP-treated tendon, similar to the staining observed in untreated normal tendon.

FIG. 8. Immunohistochemical staining against P-Selectin and Ki-67 for PEP treated tendon. Images show immunoreactivity to Ki-67 but no reactivity for P-Selectin, indicating that all PEP exosomes had been reabsorbed by neighboring cells.

FIG. 9. An image of the MTS testing fixture. The calcaneal end of the tendon was seated in the slotted plate at the proximal end while the musculotendinous junction was clamped distally. The distal clamp was frozen with dry ice to help increase friction between the clamp and the tissue. The FDS tendon was cut prior to testing as it acted as an internal splint in-vivo but would interfere with the mechanical testing ex-vivo.

### DETAILED DESCRIPTION OF ILLUSTRATIVE EMBODIMENTS

This disclosure describes composition and methods for improving repair of damaged tendon tissues. Generally, the composition includes a purified exosome product (PEP) that is applied to damaged tendon tissue. While described herein in the context off an exemplary model of tendon repair involving the Achilles tendon, the methods described herein may be practiced to repair and/or treat any damaged tendon at any site in the body.

Tendon injuries—e.g., injuries to the Achilles tendon—can be acute (traumatic) or chronic (degenerative). Tendon healing is typically a slow process because tendon tissues tend to have a low metabolic rate, limited cellularity, and/or poor vascularity compared to tissues like muscle and bone. In addition, scars formed following tendon injury and repair are usually mechanically inferior to native tendon, which can lead to re-rupture, persistent pain, and/or decreased functional capacity, potentially leading to a delay in return to work or recreational activities for the patient.

Current clinical practice for tendon injuries involves either non-operative or operative treatment. Non-operative treatments of, for example, an Achilles tendon tear include functional bracing or casting in a resting equinus position with early range of motion and weight bearing protocols. Operative treatment varies, but in general involves sutured repair followed by 4-6 weeks of immobilization in slight plantarflexion followed by a variety of early range of motion and weight bearing protocols. While non-operative treatment can have equivalent functional outcomes compared to operative treatment, non-operative treatment has repeatedly shown to have higher rates of re-rupture. In both operative and non-operative treatment options, the tendon heals via an extrinsic process that favors adhesion formation, as described in greater detail below.

Tendon healing can be intrinsic, extrinsic, or a combination of the two. Intrinsic healing (healing from within the tendon) provides superior outcomes in terms of mechanical strength and is more analogous histologically to native tissue. Extrinsic healing, by definition, requires cells to migrate to the injury site from the surrounding tendon sheath or soft tissue. Tissues undergoing primarily extrinsic healing have shown a higher rate of re-rupture due to a higher amount of immature fibers and type III collagen. The increased ratio of type III collagen to type II collagen within the newly formed tissue typically reduces the mechanical strength of the tendon and therefore increases the risk of re-rupture. Current methods for treating tendon injuries focus on accelerating extrinsic healing and optimizing early motion protocols. Although these have shown to be beneficial, they do not address the underlying issue of poor inherent intrinsic healing.

PEP is a purified exosome product prepared using a cryodesiccation step that produces a product having a structure that is distinct from exosomes prepared using conventional methods. For example, PEP typically has a spherical or spheroidal structure rather than a crystalline structure. The spherical or spheroid exosome structures generally have a diameter of no more than 300 nanometers (nm). Typically, a PEP preparation contains spherical or spheroid exosome structures that have a relatively narrow size distribution. In some preparations, PEP includes spherical or spheroidal exosome structures with a mean diameter of 110 nm±90 nm, with most of the exosome structures having a mean diameter of 110 nm±50 nm such as, for example, 110 nm±30 nm.

An unmodified PEP preparation—i.e., a PEP preparation whose character is unchanged by sorting or segregating populations of exosomes in the preparation-naturally includes a mixture of CD63⁻ and CD63⁻ exosomes. Because CD63⁻ exosomes can inhibit unrestrained cell growth, an unmodified PEP preparation that naturally includes CD63⁻ and CD63⁻ exosomes can both stimulate cell growth for wound repair and/or tissue regeneration and limit unrestrained cell growth.

Further, by sorting CD63⁻ exosomes, one can control the ratio of CD63⁺ exosomes to CD63⁻ exosomes in a PEP product by removing CD63⁻ exosomes from the naturally-isolated PEP preparation, then adding back a desired amount of CD63⁻ exosomes. In one or more embodiments, a PEP preparation can have only CD63⁻ exosomes.

In one or more embodiments, a PEP preparation can have both CD63⁺ exosomes and CD63⁻ exosomes. The ratio of CD63⁺ exosomes to CD63⁻ exosomes can vary depending, at least in part, on the quantity of cell growth desired in a particular application. For example, a CD63⁻/CD63⁻ exosome ratio provides desired cell growth induced by the CD63⁺ exosomes and inhibition of cell growth provided by the CD63⁻ exosomes achieved via cell-contact inhibition. In certain scenarios, such as in tissues where non-adherent cells exist (e.g., blood derived components), this ratio may be adjusted to provide an appropriate balance of cell growth or cell inhibition for the tissue being treated. Since cell-to-cell contact is not a cue in, for example, tissue with non-adherent cells, one may reduce the CD63⁻ exosome ratio to avoid uncontrolled cell growth. Conversely, if there is a desire to expand out a clonal population of cells, such as in allogeneic cell-based therapy or immunotherapy, one can increase the ratio of CD63⁻ exosomes to ensure that a large population of cells can be derived from a very small source.

Thus, in one or more embodiments, the ratio of CD63⁻ exosomes to CD63⁻ exosomes in a PEP preparation may be at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, or at least 16:1. In one or more embodiments, the ratio of CD63⁺ exosomes to CD63⁻ exosomes in a PEP preparation may be at most 15:1, at most 16:1, at most 17:1, at most 18:1, at most 19:1, at most 20:1, at most 25:1, or at most 30:1. For example, the ratio of CD63⁻ exosomes to CD63⁻ exosomes may be between 1:1 to 30:1, 2:1 to 20:1, 4:1 to 15:1, or 8:1 to 10:1. In one or more certain embodiments, the PEP product is formulated to contain a 9:1 ratio of CD63⁺ exosomes to CD63⁻ exosomes. In one or more certain embodiments, native PEP, e.g., PEP with an unmodified ratio of CD63 exosomes to CD63⁻ exosomes may be used.

Production of purified exosome product (PEP) involves separating plasma from blood, isolating a solution of exosomes from separated plasma with filtration and centrifugation. PEP is fully characterized and methods for preparing PEP are described in International Patent Application No. PCT/US2018/065627 (published as International Publication No. WO 2019/118817), U.S. Patent Publication No. 2021/0169812 A1, and U.S. Pat. No. 10,596,123, each of which is incorporated by reference herein in its entirety.

### In Vivo Experiments

The compositions and methods described herein may be measured for efficacy in tendon repair using any suitable animal model. As discussed herein, the described compositions and methods have been shown to be efficacious in treatment of a rabbit model of Achilles tenotomy. However, any suitable animal mode, such as mouse, rat, horse, pig, or primate may be used. Additionally, the model of tendon repair is not limited to Achilles tenotomy. Any suitable model of tendon rupture may be used.

### Surgical Technique

Rabbits were divided into three groups. In Group 1, an Achilles tenotomy was performed followed by standard suture repair. In Group 2, an Achilles tenotomy was performed, followed by standard suture repair with a type I collagen scaffold applied at the repair site. In Group 3, an Achilles tenotomy was performed, followed by standard suture repair with a type I collagen scaffold loaded with 20% PEP applied at the repair site.

Forty-four (98%) rabbits survived to their respective endpoints. One rabbit was euthanized on post-operative day 8 due to pain and during autopsy was found to have a contralateral dislocated patella. Eighteen (40%) rabbits required cast revisions (12 for slippage, 6 for swollen toes). Four rabbits from Group 2 were found to have post-operative hematuria on post-operative day 1 which self-resolved. These four rabbits were part of a group of nine rabbits from Group 2 operated on the same day. Average weight loss was 0.21+0.14 grams without any significant difference between treatment groups (p=0.49).

### Mechanical Testing

Repair of a ruptured tendon may be measured by changes to the mechanical properties of the tendon, such as failure load, tensile strength, stiffness, and Young's modulus. In one or more embodiments, mechanical testing may be used to compare tendon repair progression in animals treated with different compositions, e.g., to compare animals treated with and without PEP. In one or more embodiments, the compositions and methods described herein that include PEP may improve mechanical properties of a ruptured tendon more rapidly and/or more completely compared to compositions and methods that do not include PEP.

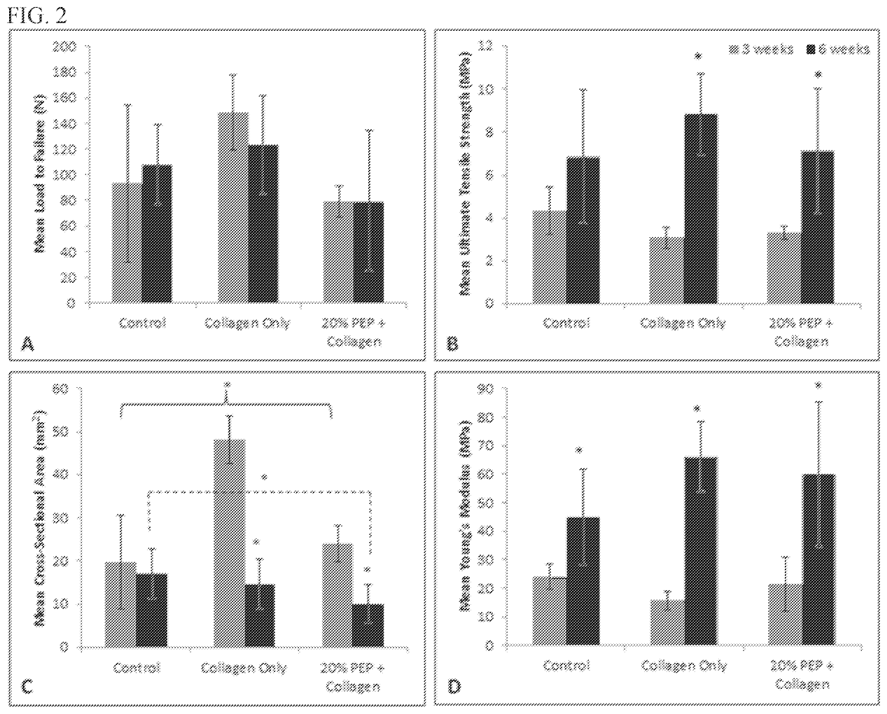

The failure load and ultimate tensile strength were found to be similar (p>0.15) across all groups, although the tensile strength at six weeks was significantly higher than that at three weeks in collagen and collagen+PEP groups (p<0.05) (Table 2, FIG. 2A, B). The cross-sectional area measured prior to MTS testing was found to be less (p=0.04) for specimens in Group 3 compared to Group 1 or Group 2 by the six-week time point (Table 1, FIG. 2C). The Young's modulus increased (p≤0.03) overtime for all groups (Table 1, FIG. 2D). There was a significant interaction between groups in relation to Young's modulus and cross-section al area (p=0.03) in favor of greater stiffness per cross-sectional area for PEP treated groups versus control groups. There was no significant interaction between groups with regard to ultimate tensile strength and cross-sectional area (p=0.84). The most common failure mode was at the repair site (65%, n=17) (Table 2). Other failure modes included calcaneal avulsion (n=6) and slippage at the distal tooth clamp (n=3).

### Histologic Analysis

Repair of a ruptured tendon may be measured by histological analysis of the tendon. Histological properties that may be measured include collagen fiber density, collagen fiber organization, and microscopic and macroscopic adhesion grading. In one or more embodiments, histologic analysis may be used to compare tendon repair progression in animals treated with different compositions, e.g., to compare animals treated with and without PEP. In one or more embodiments, the compositions and methods described herein that include PEP may improve histologic measures of a ruptured tendon compared to compositions and methods that do not include PEP.

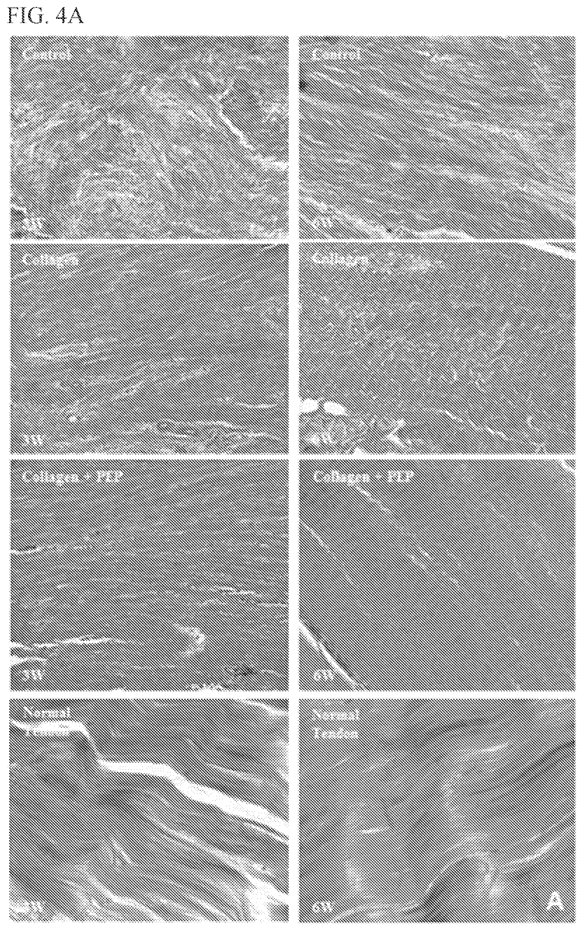

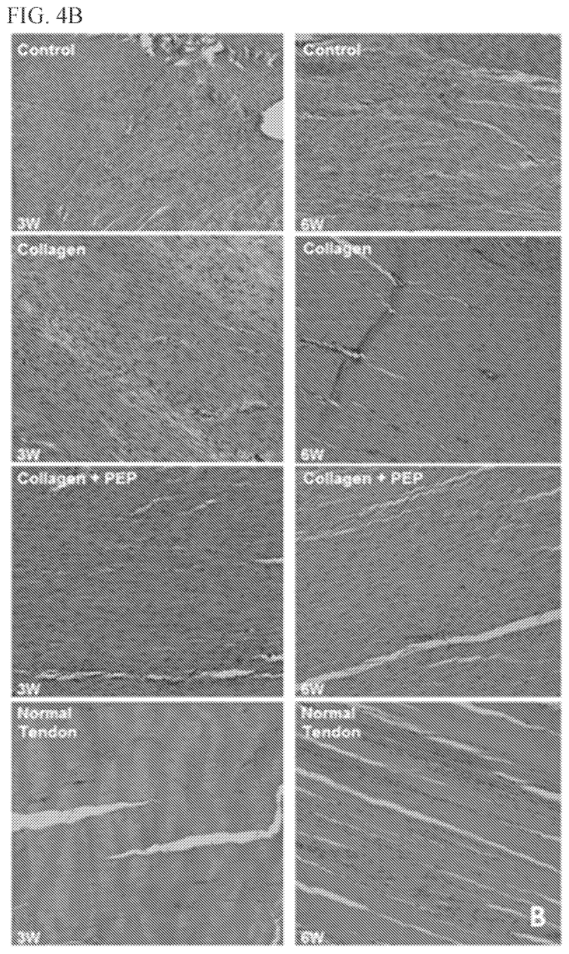

Six specimens from each group were analyzed histologically both with hematoxylin and eosin stains as well as Mason trichrome stains. Tendon treated with PEP was found to contain dense collagen fibers with parallel organization (FIG. 4) closer resembling normal tendon compared to the disorganized structure commonly found in Group 1 and Group 2. There appeared to be mature (flattened) nuclei in the PEP treated groups as well as overtime (FIG. 4) approaching the acellular-like nature of normal tendon.

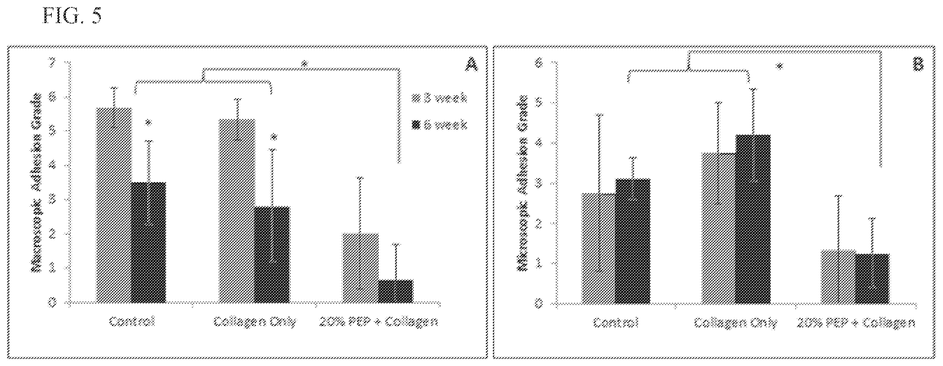

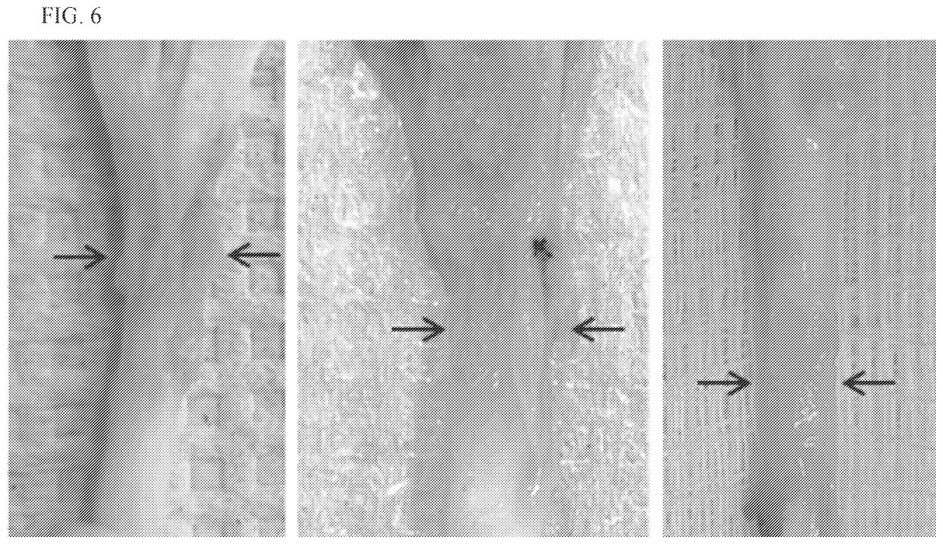

Tendon treated with PEP was found to have lower (p≤0.006) adhesion grade both macroscopically and microscopically compared to Group 1 and Group 2 (Table 3, FIG. 5, FIG. 6). Microscopic adhesion grading was performed by 3 physicians, demonstrating a mean interrater variability coefficient of −0.16.

### Immunohistochemistry

Repair of a ruptured tendon may be measured by immunohistochemical analysis of the tendon. Immunohistochemistry may be used to detect expression of certain proteins or genes associated with tendon repair. Proteins associated with tendon repair include, but are not limited to Type I collagen, Type III collagen, or Ki-67. In one or more embodiments, immunohistochemical analysis may be used to compare tendon repair progression in animals treated with different compositions, e.g., to compare animals treated with and without PEP. In one or more embodiments, the compositions and methods described herein that include PEP may improve immunohistochemical measures of a ruptured tendon during healing compared to compositions and methods that do not include PEP.

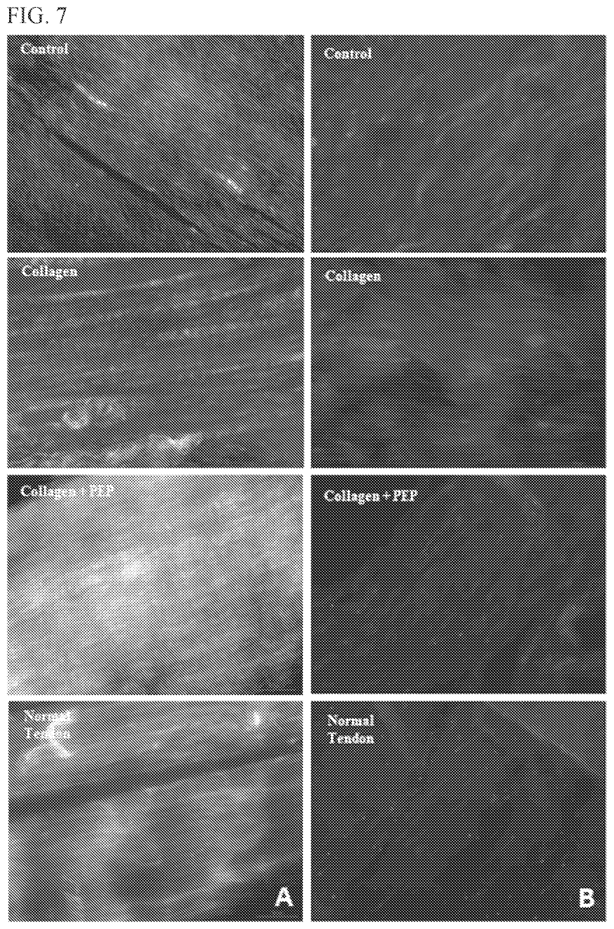

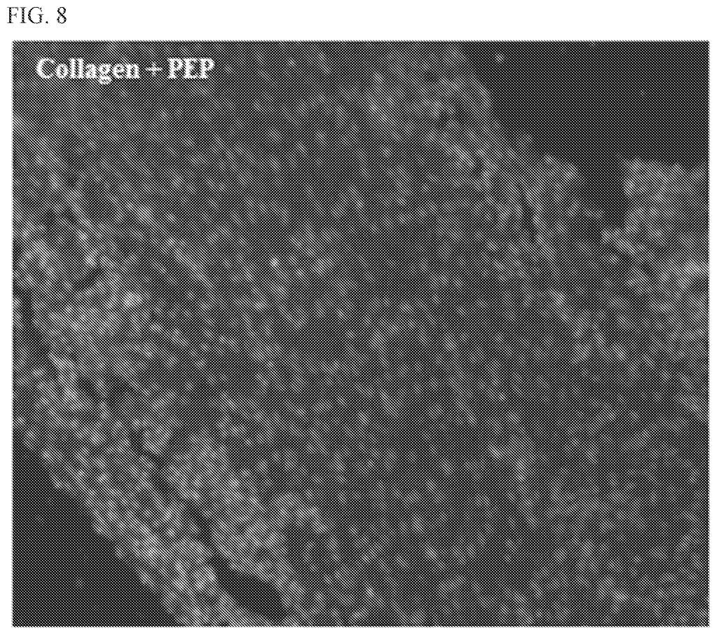

Six specimens from each group were analyzed for immunohistochemical analysis with multiple antibody combinations. After analysis under fluorescent microscopy, tendon treated with PEP was found to stain more similar to normal tendon compared to Group 1 and Group 2 with regard to ratios of Type I to Type III collagen (FIG. 7). Cellular proliferation was prominent across all groups as indicated by Ki-67 marker, while the antibody marker for PEP (P-Selectin) was not visualized at either time point in Group 3 indicating that exosomes had already been resorbed by the three-week time point (FIG. 8).

Thus, this disclosure describes treating an injured tendon using a supportive matrix (e.g., a collagen scaffold) supplemented with PEP. When compared to tendons treated with collagen or solely suture repair, treatment that included PEP provided a greater degree of intrinsic healing compared to the other groups. This finding was supported by both mechanical and histologic findings.

Native tendon has a high Young's modulus and ultimate tensile strength. Specifically, the human Achilles tendon can have an ultimate tensile strength of 100-110 MPa. Scar tissue formed by extrinsic healing has shown to produce inferior material properties such as, for example, a significant decrease in load to failure, ultimate tensile strength, and Young's Modulus at both three weeks and six weeks post-surgery in rabbit Achilles tendons treated with primary suture repair compared to normal tendon. In the current study, the failure load and ultimate tensile strength were similar across all groups despite the smaller cross-sectional diameter of the PEP-treated tendon, suggesting an increase in stiffness in the PEP-treated tendons. Additionally, there was a significant interaction between groups in relation to Young's modulus and cross-sectional area (p=0.03) in favor of greater stiffness per cross-sectional area in the PEP-treated animals and fewer adhesions were observed within the PEP-treated group. All these findings suggest a propensity for intrinsic versus extrinsic healing in the PEP treated tendon repairs.

Histologically, native tendon consists of 80% type I collagen, up to 5% type III collagen, 2% elastin, minimal cellularity, and minimal vascularity. Throughout the phases of extrinsic healing, one observes higher ratios of type III collagen, greater percentages of fibroblasts, and disorganized collagen architecture compared to native tendon. Type III collagen is present in higher percentage in scar tissue while tendon undergoing intrinsic healing has a higher ratio of type I collagen. Tendon undergoing intrinsic healing has an upregulation of type I collagen and downregulation of type III collagen. In this current study, the architecture and staining of PEP-treated tendons was more analogous to native tendon when compared to collagen-only treatments and suture-only controls. This further supports the hypothesis that PEP may promote intrinsic healing.

Extrinsic tendon healing favors adhesion formation. While adhesions support tendon repair, they can lead to complications including limitation in motion, decreased tendon gliding, and/or pain. Adhesion prevention following tendon repair has been an active area of research for several decades. Several therapies have been used to help prevent and/or treat adhesions including NSAIDs, 5-FU, and barrier sheaths, but none have completely prevented adhesion formation. In this current study, tendon treated with PEP demonstrated less macroscopic and microscopic circumferential adhesions. This finding supports the hypothesis that PEP favors intrinsic healing.

This disclosure provides evidence that PEP promotes the intrinsic healing of tendon as it decreases adhesion production, increases the ratio of type I to type III collagen, demonstrates a more organized collagen architecture while maintaining an equivalent load to failure and ultimate tensile strength. PEP is a cell-free, off-the-shelf product that can promote tendon regeneration and provides a viable solution for physicians and patients to decrease pain, improve functional capacity, and thus accelerate return to work and/or recreational activity. Given the lack of solutions for patients suffering from tendon related injury, the results herein support clinical translation of this technology in patients with chronic disabling tendon disease.

This disclosure therefore describes compositions and methods for improving repair of tendon tissue. Generally, the compositions include PEP and a pharmaceutically acceptable carrier. In a surgical setting, the PEP may be combined with a carrier that is suitable for application to tendon tissue such as, for example, a surgical glue, a tissue adhesive, and/or a supportive matrix (e.g., a collagen scaffold). As used herein, “collagen scaffold” refers to a three-dimensional network including collagen, such as a hydrogel.

Combining the PEP with collagen can increase the rate at which the reconstituted PEP forms a gel at 37° C. Indeed, the rate at which reconstituted PEP gels in the presence of collagen is influenced, at least in part, by the concentration of collagen. Increasing rates of gelation can be achieved using higher concentrations of collagen, with a maximum concentration of 10 mg/ml. In some embodiments, PEP is used in combination with collagen at a collagen concentration of 5 mg/mL. Combination with other gelling materials including thrombin glue (e.g., TISSEEL, Baxter Healthcare Corp., Deerfield, IL), hyaluronic acid, polyvinyl alcohol (PVA), poly(lactic-co-glycolic acid) (PLGA), and others. When PEP is formulated as a gel with collagen, the PEP exosomes can attach to collagen fibrils, creating a “beads on a string” appearance.

In one or more embodiments, the supportive matrix includes least one extracellular matrix component. Suitable extracellular matrix components include, but are not limited to, proteins such as collagen, elastin, fibronectin, or laminin, proteoglycans, and hyaluronic acid. In embodiments wherein the composition includes collagen, the collagen may be provided as procollagen, fibrillar collagen, such as type I collagen, type III collagen, or a combination thereof. In embodiments wherein the composition includes collagen, the collagen may be provided as a collagen scaffold. In one or more other embodiments, the extracellular matrix components may be supplied in any suitable form, such as purified recombinant protein. In one or more embodiments, the composition may include PEP and one or more supportive matrix components (e.g., collagen) in a ratio of 1:20 to 1:5 (5% v/v to 20% v/v). In one or mor alternative embodiments, the ratio of PEP to supportive matrix components may be 1:100, 1:500, 1:1000, 1:10, 1:5, 1:2, or 1:1 by volume. Any medically suitable form of collagen may be included in the composition, such as type I collagen, II collagen, or III collagen. The collagen may be derived from a mammalian source, such as a bovine or human source. The collagen fibrillar structure can be native, atelocollagen, hydrolyzed, or a combination of several types. Typically, no more than 10% of the collagen in the collagen scaffold demonstrates faster than alpha characteristics using gel electrophoresis.

Thus, the method includes administering an effective amount of the composition to tendon tissue in need of repair. In this aspect, an “effective amount” is an amount effective to decrease adhesion production, increase the ratio of type I to type III collagen, and/or produce a more organized collagen architecture compared to untreated tendon tissue or tendon tissue treated with a supportive matrix alone (no PEP). The method may improve at least one histological measure of the ruptured tendon. Exemplary histological measurements include, but are not limited to, an increase in fiber continuity, an increase in fiber parallel orientation, an increase in collagen fiber density, a decrease in vascularity, or a decrease in cellularity compared to a tendon treated without PEP.

As used herein, a “subject” can be a human or any non-human animal. Exemplary non-human animal subjects include, but are not limited to, a livestock animal or a companion animal. Exemplary non-human animal subjects include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, reindeer, etc.), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

PEP may be formulated with a pharmaceutically acceptable carrier to form a pharmaceutical composition. As used herein, “carrier” includes any solvent, dispersion medium, vehicle, coating, diluent, antibacterial, and/or antifungal agent, isotonic agent, absorption delaying agent, buffer, hydrogel, carrier solution, suspension, colloid, water and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the PEP without causing any undesirable biological effects or interacting in a deleterious manner with any of the other components of the pharmaceutical composition in which it is contained. As noted above, in a surgical setting, exemplary suitable carriers include surgical glue, tissue adhesive, or supportive matrix (e.g., a collagen scaffold).

A pharmaceutical composition containing PEP may be formulated in a variety of forms adapted to a preferred route of administration. Thus, a pharmaceutical composition can be administered via known routes including, for example, oral, parenteral (e.g., intradermal, transcutaneous, subcutaneous, intramuscular, intravenous, intraperitoneal, etc.), or topical (e.g., application to tendon tissue exposed during surgery, intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

Thus, a pharmaceutical composition may be provided in any suitable form including but not limited to a solution, a suspension, an emulsion, a spray, an aerosol, or any form of mixture. The pharmaceutical composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

Suitable excipients may include, for example, human or bovine collagen, hyaluronic acid-based compounds, human fibrinogen, or human thrombin.

The lyophilized composition including PEP may be combined with an additional excipient, which may additionally be lyophilized. Components of the lyophilized composition may be co-packaged, or may be separately provided and mixed before use to create a PEP-loaded biocompatible scaffold. The lyophilized excipient may be, for example, lyophilized human or bovine collagen, hyaluronic acid-based compounds, human fibrinogen, human thrombin, or other lyophilized powders that form a biocompatible gel when put in contact with bodily fluids (ex. blood or interstitial fluid).

In one or more embodiments, the compositions described herein are administered via injection into/onto the tendon, arthroscopically, or during open surgical repair. The compositions may be administered alone, or in addition to traditional surgical repair methods, such as sutures or staples. The product may also be used to enhance the biocompatibility and therapeutic effect of tendon sutures, anchors, patches, or other devices used to repair tendinous injures.

A formulation may be conveniently presented in unit dosage form and may be prepared by methods well known in the art of pharmacy. Methods of preparing a composition with a pharmaceutically acceptable carrier include the step of bringing the PEP into association with a carrier that constitutes one or more accessory ingredients. In general, a formulation may be prepared by uniformly and/or intimately bringing the PEP into association with a liquid carrier, a finely divided solid carrier, or both, and then, if necessary, shaping the product into the desired formulations.

The amount of PEP administered can vary depending on various factors including, but not limited to, the content and/or source of the PEP being administered, the weight, physical condition, and/or age of the subject, and/or the route of administration. Thus, the absolute weight of PEP included in a given unit dosage form can vary widely, and depends upon factors such as the species, age, weight, and physical condition of the subject, and/or the method of administration. Accordingly, it is not practical to set forth generally the amount that constitutes an amount of PEP effective for all possible applications. Those of ordinary skill in the art, however, can readily determine the appropriate amount with due consideration of such factors.

In one or more embodiments, a dose of PEP can be measured in terms of the PEP exosomes delivered in a dose. Thus, in one or more embodiments, the method can include administering sufficient PEP to provide a dose of, for example, from 1×10⁶ PEP exosomes to 1×10¹⁵ PEP exosomes to the subject, although in one or more embodiments the methods may be performed by administering PEP in a dose outside this range.

In one or more embodiments, therefore, the method can include administering sufficient PEP to provide a minimum dose of at least 1×10⁶ PEP exosomes, at least 1×10⁷ PEP exosomes, at least 1×10⁸ PEP exosomes, at least 1×10⁹ PEP exosomes, at least 1×10¹⁰ PEP exosomes, at least 1×10¹¹ PEP exosomes, at least 2×10¹¹ PEP exosomes, at least 3×10¹¹ PEP exosomes, at least 4×10¹¹ PEP exosomes, at least 5×10¹¹ PEP exosomes, at least 6×10¹¹ PEP exosomes, at least 7×10¹¹ PEP exosomes, at least 8×10¹¹ PEP exosomes, at least 9×10¹¹ PEP exosomes, at least 1×10¹² PEP exosomes, 2×10¹² PEP exosomes, at least 3×10¹² PEP exosomes, at least 4×10¹² PEP exosomes, or at least 5×10¹² PEP exosomes, at least 1×10¹³ PEP exosomes, or at least 1×10¹⁴ PEP exosomes.

In one or more embodiments, the method can include administering sufficient PEP to provide a maximum dose of no more than 1×10¹⁵ PEP exosomes, no more than 1×10¹⁴ PEP exosomes, no more than 1×10¹³ PEP exosomes, no more than 1×10¹² PEP exosomes, no more than 1×10¹¹ PEP exosomes, or no more than 1×10¹⁰ PEP exosomes.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1×10¹¹ to 1×10¹³ PEP exosomes such as, for example, a dose of from 1×10¹¹ to 5×10¹² PEP exosomes, a dose of from 1×10¹² to 1×10¹³ PEP exosomes, or a dose of from 5×10¹² to 1×10¹³ PEP exosomes. In one or more certain embodiments, the method can include administering sufficient PEP to provide a dose that is equal to any minimum dose or any maximum dose listed above. Thus, for example, the method can involve administering a dose of 1×10¹⁰ PEP exosomes, 1×10¹¹ PEP exosomes, 5×10¹¹ PEP exosomes, 1×10¹² PEP exosomes, 5×10¹² PEP exosomes, 1×10¹³ PEP exosomes, or 1×10¹⁴ PEP exosomes.

Alternatively, a dose of PEP can be measured in terms of the concentration of PEP upon reconstitution from a lyophilized state. Thus, in one or more embodiments, the methods can include administering PEP to a subject at a dose of, for example, from a 0.01% solution to a 100% solution to the subject, although in one or more embodiments the methods may be performed by administering PEP in a dose outside this range. As used herein, a 100% solution of PEP refers to one vial of PEP (2×10¹¹ exosomes or 75 mg) solubilized in 1 ml of a liquid or gel carrier (e.g., water, phosphate buffered saline, serum free culture media, surgical glue, tissue adhesive, etc.). For comparison, a dose of 0.01% PEP is roughly equivalent to a standard dose of exosomes prepared using conventional methods of obtaining exosomes such as exosome isolation from cells in vitro using standard cell conditioned media.

In one or more embodiments, therefore, the method can include administering sufficient PEP to provide a minimum dose of at least 0.01%, at least 0.05%, at least 0.1%, at least 0.25%, at least 0.5%, at least 1.0%, at least 2.0%, at least 3.0%, at least 4.0%, at least 5.0%, at least 6.0%, at least 7.0%, at least 8.0%, at least 9.0%, at least 10%, at least 15%, at least 20%, at least 25%, at least 30%, at least 35%, at least 40%, at least 45%, at least 50%, at least 60%, or at least 70%.

In one or more embodiments, the method can include administering sufficient PEP to provide a maximum dose of no more than 100%, no more than 90%, no more than 80%, no more than 70%, no more than 60%, no more than 50%, no more than 40%, no more than 30%, no more than 20%, no more than 10%, no more than 9.0%, no more than 8.0%, no more than 7.0%, no more than 6.0%, no more than 5.0%, no more than 4.0%, no more than 3.0%, no more than 2.0%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%.

In one or more embodiments, the method can include administering sufficient PEP to provide a dose characterized by a range having endpoints defined by any a minimum dose identified above and any maximum dose that is greater than the minimum dose. For example, in one or more embodiments, the method can include administering sufficient PEP to provide a dose of from 1% to 50% such as, for example, a dose of from 5% to 20%. In certain embodiments, the method can include administering sufficient PEP to provide a dose that is equal to any minimum dose or any maximum dose listed above. Thus, for example, the method can involve administering a dose of 0.05%, 0.25%, 1.0%, 2.0%, 5.0%, 20%, 25%, 50%, 80%, or 100%.

A single dose may be administered all at once, continuously for a prescribed period of time, or in multiple discrete administrations. When multiple administrations are used, the amount of each administration may be the same or different. For example, a prescribed daily dose of may be administered as a single dose, continuously over 24 hours, as two administrations, which may be equal or inequal. When multiple administrations are used to deliver a single dose, the interval between administrations may be the same or different. In one or more certain embodiments, PEP may be administered from a one-time administration, for example, during a surgical procedure. In one or more certain embodiments in which multiple administrations of the PEP composition are administered to the subject, the PEP composition may be administered as needed to regenerate the tendon tissue to the desired degree. Alternatively, the PEP composition may be administered twice, three times, four times, five times, six times, seven times, eight times, nine times, or at least ten times. The interval between administrations can be a minimum of at least one day such as, for example, at least three days, at least five days, at least seven days, at least ten days, at least 14 days, or at least 21 days. The interval between administrations can be a maximum of no more than six months such as, for example, no more than three months, no more than two months, no more than one month, no more than 21 days, or no more than 14 days.

In one or more embodiments, the method can include multiple administrations of PEP to a subject at an interval (for two administrations) or intervals (for more than two administrations) characterized by a range having endpoints defined by any a minimum interval identified above and any maximum interval that is greater than the minimum interval. For example, in one or more embodiments, the method can include multiple administrations of PEP at an interval or intervals of from one day to six months such as, for example, from three days to ten days. In one or more certain embodiments, the method can include multiple administrations of PEP at an interval of that is equal to any minimum interval or any maximum interval listed above. Thus, for example, the method can involve multiple administrations of PEP at an interval of three days, five days, seven days, ten days, 14 days, 21 days, one month, two months, three months, or six months.

In one or more embodiments, the methods can include administering a cocktail of PEP that is prepared from a variety of cell types, each cell type having a unique tendon-supporting profile—e.g., protein composition and/or gene expression. In this way, the PEP composition can provide a broader spectrum of tendon-supporting activity than if the PEP composition is prepared from a single cell type.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

### EXEMPLARY EMBODIMENTS

Embodiment 1 is a composition comprising:
purified exosome product (PEP); and
a pharmaceutically acceptable carrier comprising a supportive matrix.

Embodiment 2 is the composition of embodiment 1, wherein the PEP comprises spherical or spheroid exosomes having a diameter no greater than 300 nm.

Embodiment 3 is the composition of embodiment 1, wherein the PEP comprises spherical or spheroid exosomes having a mean diameter of 110 nm±90 nm.

Embodiment 4 is the composition of embodiment 3, wherein the PEP comprises spherical or spheroid exosomes having a mean diameter of 110 nm±50 nm.

Embodiment 5 is the composition of embodiment 4, wherein the PEP comprises spherical or spheroid exosomes having a mean diameter of 110 nm±30 nm.

Embodiment 6 is the composition of any preceding embodiment, wherein the PEP comprises:
from 1% to 20% CD63⁻ exosomes; and
from 80% to 99% CD63+exosomes.

Embodiment 7 is the composition of any one of embodiments 1-5, wherein the PEP comprises at least 50% CD63⁻ exosomes.

Embodiment 8 is the composition of any preceding embodiment, wherein the PEP comprises from 1×10¹¹ PEP exosomes to 1×10¹³ PEP exosomes.

Embodiment 9 is the composition of embodiment 8, wherein the PEP comprises from 1×10¹² PEP exosomes to 1×10¹³ PEP exosomes.

Embodiment 10 is the composition of any preceding embodiment, wherein the supportive matrix comprises a collagen scaffold.

Embodiment 11 is the composition of embodiment 10, wherein the collagen scaffold comprises type I fibrillar collagen.

Embodiment 12 is a method of treating injured tendon tissue, the method comprising applying the composition of any preceding embodiment to injured tendon tissue.

Embodiment 13 is the method of embodiment 12, wherein the composition is applied in an amount effective to decrease adhesion production compared to injured tendon tissue treated without the composition.

Embodiment 14 is the method of embodiment 12, wherein the composition is applied in an amount effective to increase the ratio of type I to type III collagen compared to injured tendon tissue treated without the composition.

Embodiment 15 is the method of embodiment 12, wherein the composition is applied in an amount effective to produce more organized collagen architecture compared to injured tendon tissue treated without the composition.

Embodiment 16 is the method of any one of embodiments 12-15, wherein the injured tendon tissue comprises disruption of a tendon.

Embodiment 17 is the method of embodiment 16, wherein the disruption of the tendon comprises rupture of the tendon.

Embodiment 18 is the method of embodiment 17, wherein the rupture of the tendon comprises an Achilles tendon rupture.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

In this Example, a rabbit model of Achilles tenotomy was used to study the impact of collagen or collagen and PEP on tendon repair.

### Study Design

The current study was performed in young adult (10-12 weeks) New Zealand White female rabbits with weights ranging from 2.7 kg to 3.0 kg. The study design included three groups of 15 rabbits, for a total of 45 rabbits. Endpoints were at three weeks and six weeks post-surgery, based on predicate studies showing histological differences between these time points. The transition from Type III collagen to Type I collagen does not begin until the remodeling stage (6-10 weeks), which supports the selected time points.

Group 1 was the control group where an Achilles tenotomy was performed followed by standard suture repair. Group 2 underwent an Achilles tenotomy followed by standard suture repair with a type I collagen scaffold applied at the repair site. Group 3 underwent an Achilles tenotomy followed by standard suture repair with a type I collagen scaffold loaded with 20% PEP applied at the repair site. For each rabbit, only one Achilles tendon was operated on.

Three weeks following surgery, six rabbits in each group were sedated and sacrificed via intravenous injection of Fatal Plus (Vortech Pharmaceuticals, Ltd., Dearborn, MI). In each group, three specimens were used for histologic testing and the remaining three specimens were used for mechanical testing. The non-operative contralateral Achilles tendons also were collected for comparative analysis. Six weeks following surgery, the remaining nine rabbits in each group were sacrificed. In each group, three specimens were used for histologic testing and the remaining six specimens were used for mechanical testing. All rabbits who survived the entire assigned time point (three weeks or six weeks) were included for data analysis.

### Scaffold Preparation

The scaffolds utilized for Group 2 and Group 3 were made from research grade type I fibrillar pH neutral collagen (50 mg/mL, Collagen Solutions, Inc., Eden Prairie, MN). The scaffold for Group 2 was made of collagen only, while the scaffold for Group 3 was combined with PEP (Rion LLC, Rochester, MN) to achieve a PEP exosome concentration of 1×10¹² exosome/mL. PEP has shown to contain fibroblast growth factor 2 (FGF-2), platelet derived growth factor BB (PDGF-BB), insulin-like growth factor 1 (IGF-1), and transforming growth factor beta (TGF-β). The PEP used in these experiments was native PEP, meaning that the ratio of CD63⁺ exosomes to CD63⁻ exosomes had not been modified.

The scaffold preparation technique was designed so that it could be performed via a sterile technique intraoperatively to mimic the anticipated clinical scenario. For Group 2, the scaffolds were created using an 80:20 ratio of type I fibrillar collagen (Collagen Solutions, Inc., Eden Prairie, MN) and normal saline. For Group 3, the type I fibrillar collagen (Collagen Solutions, Inc., Eden Prairie, MN) was mixed with PEP at a ratio of 80:20. This provided a scaffold that had a paste consistency, allowing it to be applied over the repaired tenotomy site.

### Surgical Procedure

Approval was obtained for this study from the Institutional Animal Care and Use Committee (IACUC). Rabbits were divided into three groups. In Group 1, an Achilles tenotomy was performed followed by standard suture repair. In Group 2, an Achilles tenotomy was performed, followed by standard suture repair with a type I collagen scaffold applied at the repair site. In Group 3, an Achilles tenotomy was performed, followed by standard suture repair with a type I collagen scaffold loaded with 20% PEP applied at the repair site.

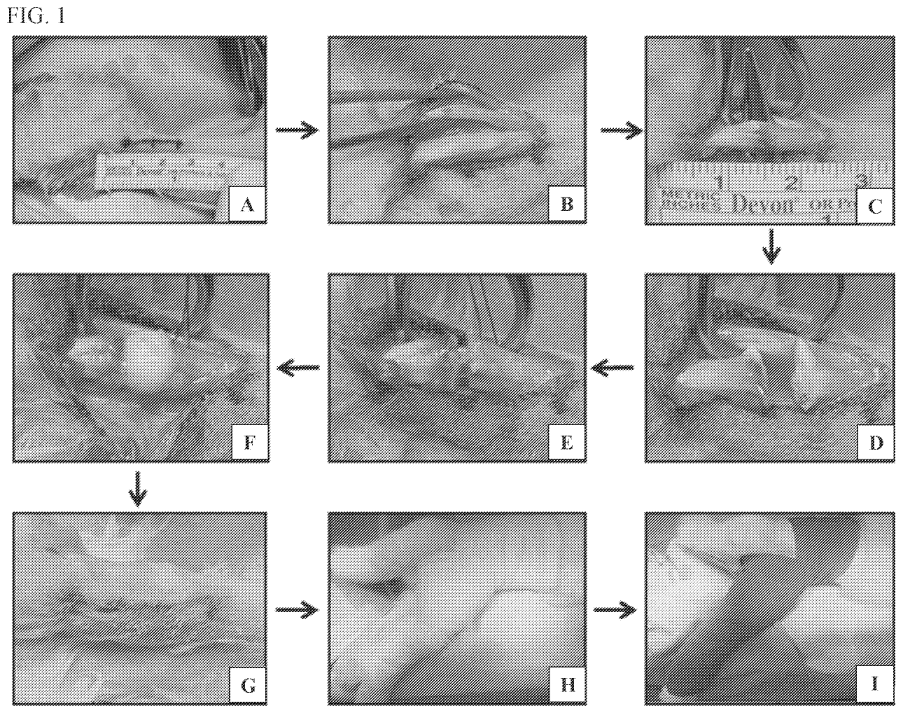

On the day of surgery, the rabbits received pre-operative antibiotics and analgesia. The rabbits were anesthetized with inhaled isoflurane gas, which was provided throughout the entire procedure. The Achilles tenotomy was performed through a 2-cm longitudinal incision that was marked starting 0.5 cm proximal to the calcaneal tubercle (FIG. 1A). Dissection was performed down to the flexor digitorum superficialis (FDS). The paratenon surrounding the FDS was incised. The FDS was isolated and retracted laterally to expose the Achilles tendon (FIG. 1B). The Achilles tendon bundle was isolated and tenotomized 1.5 cm proximal to the calcaneal tubercle (FIG. 1C, D). Care was taken not to cut the FDS as the FDS was to act as an internal splint for the repair site. The two ends of the tendon were then repaired in 150° of plantarflexion utilizing a modified Kessler core suture technique (FIG. 1E). Minimal suture repair was desired as the goal was to assess the effects of the scaffold rather than the strength of the suture repair, but the suture would help prevent initial gap formation. Suture repair was performed in the same fashion across all groups with a 5-0 polydioxanone suture (PDS, Ethicon, Inc., Raritan, NJ) to allow for clean histologic assessment of specimens. In Group 2 and Group 3 0.2 mL of scaffold was placed topically at the tenotomy site prior to final tightening of the suture repair (FIG. 1F). The solution rapidly gelled after application. The paratenon was not repaired. Skin was closed with 3-0 absorbable vicryl suture (Ethicon, Inc., Raritan, NJ) (FIG. 1G). The hindlimb was placed in a hip-spica-like cast from the toes to high into the groin, molding the ankle at 150° of plantarflexion (FIG. 1H). The cast was then overwrapped with vet wrap from the toes up the leg and figure-eight wrapping around the abdomen (FIG. 1I).

Forty-four (98%) rabbits survived to their respective endpoints. One rabbit was euthanized on post-operative day 8 due to pain and during autopsy was found to have a contralateral dislocated patella. Eighteen (40%) rabbits required cast revisions (12 for slippage, 6 for swollen toes). Four rabbits from Group 2 were found to have post-operative hematuria on post-operative day 1 which self-resolved. These four rabbits were part of a group of nine rabbits from Group 2 operated on the same day. Average weight loss was 0.21+0.14 grams without any significant difference between treatment groups (p=0.49).

Six rabbits from each group were sacrificed three weeks following surgery. In each group, three rabbits were used for histologic testing, and three rabbits were used for mechanical testing. Following sacrifice of the rabbit, the Achilles tendon was dissected from the hind limb 3 cm proximal to the myotendinous junction and sawed off distally at the calcaneal tubercle, assuring a 1 cm×1 cm bone block distally. The FDS was removed from the specimen as it would interfere with mechanical results. The remaining nine rabbits in each group were sacrificed six weeks following surgery. In each group, three rabbits were used for histologic testing, and the remaining six were used for mechanical testing. All rabbits who survived the entire assigned time point (3 or 6 weeks) were included for data analysis.

### Mechanical Testing

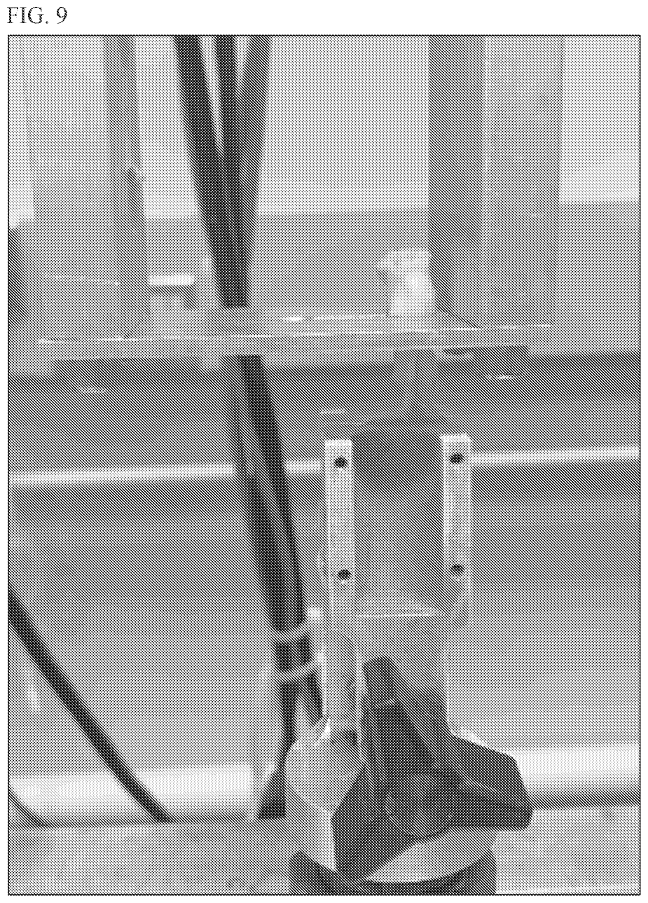

Testing was performed on a servo-hydraulic testing machine (MTS Systems, Corp., Eden Prairie, MN). The MTS fixture setup consisted of a toothed clamp distally and a slotted plate for the bone block proximally (FIG. 9). The distal clamp was enhanced with dry ice to increase friction between the clamp and the specimen. Prior to each test, the cross-sectional area of each specimen was measured as well as the initial length to determine strain and stiffness. The ultimate tensile strength, method of failure, location of failure, stress, strain, and Young's modulus were recorded for all operative and multiple non-operative samples.

The failure load and ultimate tensile strength were found to be similar (p>0.15) across all groups, although the tensile strength at six weeks was significantly higher than that at three weeks in collagen and collagen+PEP groups (p<0.05) (Table 2, FIG. 2A, B). The cross-sectional area measured prior to MTS testing was found to be less (p=0.04) for specimens in Group 3 compared to Group 1 or Group 2 by the six-week time point (Table 1, FIG. 2C). The Young's modulus increased (p<0.03) overtime for all groups (Table 1, FIG. 2D). There was a significant interaction between groups in relation to Young's modulus and cross-section al area (p=0.03) in favor of greater stiffness per cross-sectional area for PEP treated groups versus control groups. There was no significant interaction between groups with regard to ultimate tensile strength and cross-sectional area (p=0.84). The most common failure mode was at the repair site (65%, n=17) (Table 2). Other failure modes included calcaneal avulsion (n=6) and slippage at the distal tooth clamp (n=3).

TABLE 1
Summary of mechanical data performed for all groups at both 3 and 6 weeks.

| | | Group 1 | | Group 2 | | Group 3 | | |
|---|---|---|---|---|---|---|---|---|
| | | Control | | Collagen only | | Collagen + PEP | | p-value |
| Mean load to | 3 wk | 93.4 | p = 0.73 | 148.3 | p = 0.34 | 79.0 | p = 0.99 | p = 0.16 |
| failure (N) | 6 wk | 108.0 | | 123.4 | | 79.4 | | p = 0.25 |
| Mean cross- | 3 wk | 19.8 | p = 0.71 | 48.2 | p = 0.0007 | 24.0 | p = 0.008 | p = 0.007 |
| sectional area | 6 wk | 17.0 | | 14.5 | | 10.0 | | p = 0.04 |
| (mm²) | | | | | | | | |
| Stiffness | 3 wk | 16.9 | p = 0.30 | 18.0 | p = 0.20 | 19.0 | p = 0.97 | p = 0.95 |
| (N/mm) | 6 wk | 26.3 | | 26.2 | | 18.7 | | p = 0.37 |
| Mean ultimate | 3 wk | 4.34 | p = 0.12 | 3.07 | p = 0.0014 | 3.31 | p = 0.02 | p = 0.15 |
| tensile strength | 6 wk | 6.86 | | 8.83 | | 7.12 | | p = 0.46 |
| (MPa) | | | | | | | | |
| Mean Young's | 3 wk | 23.9 | p = 0.03 | 15.6 | p = 0.0004 | 21.5 | p = 0.0014 | p = 0.32 |
| modulus | 6 wk | 45.0 | | 66.0 | | 59.9 | | p = 0.21 |
| (MPa) | | | | | | | | |

TABLE 2
Mechanical data from each specimen including failure mode.

| | | | Cross | | Ultimate | | |
|---|---|---|---|---|---|---|---|
| | | Load to | Sectional | | Tensile | Young's | |
| | | Failure | Area | Stiffness | Stress | Modulus | |
| Group | Endpoint | (N) | (mm²) | (N/mm) | (MPa) | (Mpa) | Failure Mode |
| Control | 3 wks | 122.44 | 23.80 | 15.87 | 5.14 | 28.71 | calcaneal |
| | | | | | | | avulsion |
| Control | 3 wks | 134.70 | 28.08 | 28.71 | 4.80 | 23.09 | tenotomy site |
| Control | 3 wks | 23.06 | 7.48 | 6.05 | 3.08 | 19.96 | calcaneal |
| | | | | | | | avulsion |
| Collagen | 3 wks | 115.05 | 44.40 | 9.98 | 2.59 | 12.29 | distal clamp |
| Collagen | 3 wks | 168.42 | 54.56 | 18.07 | 3.09 | 15.74 | distal clamp |
| Collagen | 3 wks | 161.53 | 45.60 | 25.83 | 3.54 | 18.68 | distal clamp |
| Collagen + PEP | 3 wks | 70.29 | 19.61 | 22.15 | 3.58 | 32.47 | tenotomy site |
| Collagen + PEP | 3 wks | 93.02 | 27.88 | 16.00 | 3.34 | 15.79 | tenotomy site |
| Collagen + PEP | 3 wks | 73.66 | 24.40 | 18.79 | 3.02 | 16.18 | tenotomy site |
| Control | 6 wks | 126.43 | 27.29 | 29.27 | 4.63 | 31.20 | calcaneal |
| | | | | | | | avulsion |
| Control | 6 wks | 106.57 | 17.77 | 23.15 | 6.00 | 37.89 | tenotomy site |
| Control | 6 wks | 100.42 | 18.36 | 15.33 | 5.47 | 25.83 | calcaneal |
| | | | | | | | avulsion |
| Control | 6 wks | 78.39 | 12.24 | 16.76 | 6.40 | 44.76 | calcaneal |
| | | | | | | | avulsion |
| Control | 6 wks | 77.15 | 13.95 | 29.95 | 5.53 | 64.93 | tenotomy site |
| Control | 6 wks | 159.32 | 12.14 | 43.47 | 13.12 | 65.60 | calcaneal |
| | | | | | | | avulsion |
| Collagen | 6 wks | 122.09 | 11.60 | 25.54 | 10.53 | 78.02 | tenotomy site |
| Collagen | 6 wks | 91.50 | 14.14 | 23.75 | 6.47 | 60.73 | tenotomy site |
| Collagen | 6 wks | 172.39 | 23.94 | 34.57 | 7.20 | 51.30 | tenotomy site |
| Collagen | 6 wks | 149.96 | 14.70 | 25.28 | 10.20 | 59.92 | tenotomy site |
| Collagen | 6 wks | 81.26 | 8.32 | 21.83 | 9.77 | 79.88 | tenotomy site |
| Collagen + PEP | 6 wks | 157.67 | 13.76 | 36.31 | 11.46 | 88.76 | tenotomy site |
| Collagen + PEP | 6 wks | 61.68 | 10.08 | 17.01 | 6.12 | 62.87 | tenotomy site |
| Collagen + PEP | 6 wks | 13.34 | 3.20 | 3.11 | 4.17 | 23.59 | tenotomy site |
| Collagen + PEP | 6 wks | 122.68 | 15.51 | 29.63 | 7.91 | 73.05 | tenotomy site |
| Collagen + PEP | 6 wks | 89.74 | 9.90 | 18.69 | 9.07 | 76.13 | tenotomy site |
| Collagen + PEP | 6 wks | 31.22 | 7.83 | 7.74 | 3.99 | 34.88 | tenotomy site |

### Histologic Analysis

Tendons for the sacrificed rabbit specimens were then placed in 10% formalin solution prior to embedding in paraffin. Tissue samples were cut longitudinally between 8-10 μm with a rotating microtome (Cryocut 1800; Leica Microsystems, Inc., Buffalo Grove, IL) and fixed on glass slides. Following deparaffinization, specimens were stained with hematoxylin and eosin (H&E) to assess cellularity as well as Mason's trichrome to assess collagen content and organization. All slides were analyzed under light microscopy (Olympus DP25; Olympus America, Melville, NY) and digital images were obtained (cellSens version 1.9, Olympus America, Melville, NY). Tendon structure, collagen density and cellularity were characterized from these stains. Tendon was graded both macroscopically and microscopically for adhesions using a validated adhesion grading scale.

Six specimens from each group were analyzed histologically both with hematoxylin and eosin stains as well as Mason trichrome stains. Tendon treated with PEP was found to contain dense collagen fibers with parallel organization (FIG. 4) closer resembling normal tendon compared to the disorganized structure commonly found in Group 1 and Group 2. There appeared to be mature (flattened) nuclei in the PEP treated groups as well as overtime (FIG. 4) approaching the acellular-like nature of normal tendon.

Tendon treated with PEP was found to have lower (p≤0.006) adhesion grade both macroscopically and microscopically compared to Group 1 and Group 2 (Table 3, FIG. 5, FIG. 6). Microscopic adhesion grading was performed by three physicians, demonstrating a mean interrater variability coefficient of −0.16.

TABLE 3
Adhesion grading of tendon repair

| | | | Group 3 | p-value |
|---|---|---|---|---|
| | Group 1 | Group 2 | (Collagen + | between |
| | (Control) | (Collagen) | PEP) | groups |
| Macroscopic | 4.2 (3.1-5.3) | 3.8 (2.6-4.9) | 1.2 (0.2-2.2) | 0.0006 |
| Microscopic | 3.2 (2.1-4.3) | 3.6 (2.6-4.5) | 1.2 (0.1-2.2) | 0.0062 |

### Immunohistochemistry

Specimens were prepared, paraffin embedded, cut, and deparaffinized. Antigen retrieval was not performed as it was found to significantly alter the integrity of the tissue. Primary and secondary antibodies were applied in multiple combinations. Primary antibodies included anti-type I collagen (mouse monoclonal, 1:400; AB90395, Abcam, Cambridge, MA), anti-type III collagen (goat polyclonal, 1:400; Southern Biotech, Birmingham, AL), Ki-67 (mouse monoclonal, 1:100; Novus Biologicals, Centennial, CO), and P-Selectin (sheep polyclonal, 1:100; R&D Systems, Minneapolis, MN). Secondary antibodies included Cy3 (goat anti-mouse polyclonal, 1:100; A10521, Invitrogen, Carlsbad, CA), Alexa Fluor 680 (donkey anti-sheep polyclonal, 1:100; A21102, Invitrogen), Alexa Fluor 555 (donkey anti-goat polyclonal, 1:400; ab150130, Invitrogen) and Alexa Fluor 647 (goat anti-mouse polyclonal, 1:400; ab150115, Invitrogen) (Table 1). Slides were mounted with a DAPI enhanced glue (ProLong Gold, Invitrogen). Slides were analyzed under a contrast fluorescent microscope (Axio Observer Z1, Carl Zeiss Microscopy, Thornwood, NY) using 25× magnification. The stain intensity of Type I and Type III collagen, cellular proliferation, and presence of PEP was characterized from these slides.

Six specimens from each group were analyzed for immunohistochemical analysis with multiple antibody combinations. After analysis under fluorescent microscopy, tendon treated with PEP was found to stain more similar to normal tendon compared to Group 1 and Group 2 with regard to ratios of Type I to Type III collagen (FIG. 7). Cellular proliferation was prominent across all groups as indicated by Ki-67 marker, while the antibody marker for PEP (P-Selectin) was not visualized at either time point in Group 3 indicating that exosomes had already been resorbed by the three-week time point (FIG. 8).

### Sample Size Justification

The sample size justification was based on the biomechanical outcome of failure load with variability estimates obtained from similar studies. Predicting a similar variability in the current study, sample sizes of n=3 per group at three wks and n=6 per group at six wks would provide 80% power to detect differences between any two of the three study groups of at least 100N and 171N, respectively. More rabbits were chosen for biomechanical testing than for histology analysis at six wks due to the expected increase in variability so the increase in sample size would decrease the variability.

### Statistical Analysis

The statistical analysis focused primarily on comparing the three study groups separately at each of the two time points. Data comprised of continuous variables was analyzed using one-way analysis of variance (ANOVA). If the overall F-test was significant, further analysis was conducted using an appropriate multiple comparisons procedure to maintain the overall type I error rate. Categorical data was analyzed using chi-square tests. Interaction analysis was performed between on groups when looking at Young's modulus and cross-sectional area as well as ultimate tensile strength and cross-sectional area. All statistical tests were two-sided and p-values less than 0.05 were considered statistically significant.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “approximately” or “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition comprising:
purified exosome product (PEP); and
a pharmaceutically acceptable carrier comprising a supportive matrix.

2. The composition of claim 1, wherein the PEP comprises spherical or spheroid exosomes having a diameter no greater than 300 nm.

3. The composition of claim 1, wherein the PEP comprises spherical or spheroid exosomes having a mean diameter of 110 nm±90 nm.

4. The composition of claim 3, wherein the PEP comprises spherical or spheroid exosomes having a mean diameter of 110 nm±50 nm.

5. The composition of claim 4, wherein the PEP comprises spherical or spheroid exosomes having a mean diameter of 110 nm±30 nm.

6. The composition of claim 1, wherein the PEP comprises:
from 1% to 20% CD63⁻ exosomes; and
from 80% to 99% CD63⁺ exosomes.

7. The composition of claim 1, wherein the PEP comprises at least 50% CD63 exosomes.

8. The composition of claim 1, wherein the PEP comprises from 1×10¹¹ PEP exosomes to 1×10¹³ PEP exosomes.

9. The composition of claim 8, wherein the PEP comprises from 1×10¹² PEP exosomes to 1×10¹³ PEP exosomes.

10. The composition of claim 1, wherein the supportive matrix comprises a collagen scaffold.

11. The composition of claim 10, wherein the collagen scaffold comprises type I fibrillar collagen.

12. A method of treating injured tendon tissue, the method comprising applying the composition of claim 1 to injured tendon tissue.

13. The method of claim 12, wherein the composition is applied in an amount effective to decrease adhesion production compared to injured tendon tissue treated without the composition.

14. The method of claim 12, wherein the composition is applied in an amount effective to increase the ratio of type I to type III collagen compared to injured tendon tissue treated without the composition.

15. The method of claim 12, wherein the composition is applied in an amount effective to produce more organized collagen architecture compared to injured tendon tissue treated without the composition.

16. The method of claim 12, wherein the injured tendon tissue comprises disruption of a tendon.

17. The method of claim 16, wherein the disruption of the tendon comprises rupture of the tendon.

18. The method of claim 17, wherein the rupture of the tendon comprises an Achilles tendon rupture.
